(19) United States
(12) Patent Application Publication
(10) Pub. No.: US 2023/0032908 A1
(43) Pub. Date: Feb. 2, 2023

(54) APPARATUS AND METHOD FOR EDUCATING, INCENTIVIZING, AND MANAGING HEALTHY FOOD CHOICES BASED ON INDIVIDUAL'S BIOMETRICS AND HEALTH PROFILES

(71) Applicant: Trio Effect, LLC, Carver, MN (US)

(72) Inventors: Kara Jaehnert, Carver, MN (US); Perry Schmidt, Wayzata, MN (US)

(21) Appl. No.: 17/877,028

(22) Filed: Jul. 29, 2022

Related U.S. Application Data
(60) Provisional application No. 63/227,583, filed on Jul. 30, 2021.

(51) Int. Cl. G09B 19/00; A23L 33/00

(52) U.S. Cl. CPC G09B 19/0092; A23L 33/30; A23V 2002/00

(57) ABSTRACT

A method for monitoring and incentivizing food purchasing includes presenting a prompt for regarding a first food product code, receiving an input regarding the first food product and rating the healthiness of the first food product. The computerized method does the same for a second food product. A healthiness rating is determined for each of the first food item, the second food item and the combination of the food items. The healthiness rating includes a biometric input for setting a calorie budget and a health profile for determining the best mix of food nutrient values for a particular health profile. Incentives are provided based on the healthiness rating of the combination. A computer system can be employed to implement the above method. An instruction set stored in memory implements the method and causes the computer hardware and software to implement the method.

This application is a claims priority to U.S. Provisional Application No. 63/227,583, filed on Jul. 30, 2021, which is incorporated by reference in its entirety.

### BACKGROUND OF THE INVENTION

Currently, healthcare professionals including physicians and registered dietitians provide substantial guidance and education to patients regarding managing their medical conditions via diet. Generally, this guidance is manifested in rules-based systems with lists of items to avoid and others to emphasize in their diets. However, few tools exist to help patients be successful in adhering to these recommendations. Many patients will judge their adherence to their physician's dietary guidance on a pass-fail basis. Consequently, less than perfect compliance may be deemed a failure and could lead to greater non-compliance, further diminishing the patient's health. Furthermore, the rules-based systems are often rigid and categorizes foods as being “good” or “bad”, when reality is much more nuanced. A careful consideration of food's nutrients is necessary to properly evaluate the appropriateness of that food given a patient's medical conditions. Unfortunately, this is a burdensome, impractical process that many patients forgo. While there are numerous calorie trackers in the marketplace, there is no method or process available today in the marketplace that leverages an individuals' personal biometrics and health profile concurrently with the process of purchasing food. Furthermore, there are no methods or processes available in the marketplace that will help users analyze the nutrient content their food and evaluate the appropriateness of the food items based on their health profile.

### BRIEF DESCRIPTION OF THE DRAWINGS

FIG. 1 is a method for incentivizing food consumers to make better food shopping choices, according to an example embodiment.

FIG. 2 is a method for incentivizing food consumers to make better food shopping choices, according to an example embodiment.

FIG. 3 is a schematic view of a computer system for determining compensation based on food choices, according to an example embodiment.

FIG. 4 shows a diagrammatic representation of a computing device for a machine in the example electronic form of a computer system, according to an example embodiment.

FIG. 5 is a schematic drawing of a machine-readable medium that includes an instruction set, according to an example embodiment.

FIG. 6 is a computer system in the form of a smartphone that includes a touch screen display a keyboard, according to an example embodiment.

FIG. 7 shows a diagrammatic representation of a system for scoring a collection of food products, according to an example embodiment.

FIG. 8 is a flow diagram of a method for scoring a combination of foods, according to another embodiment of the invention.

FIG. 9 is a flow diagram for another method 900 of monitoring a collection of a plurality of foods, according to yet another embodiment.

FIG. 10 is another schematic diagram of a computer system, according to an example embodiment.

FIG. 11 is a flow diagram for data used by a computer system, according to an example embodiment.

### DETAILED DESCRIPTION OF THE INVENTION

Disclosed herein is a program designed to help a consumer or food purchaser make better or healthier food purchasing decisions. The method can be employed without a computer or can be computer based. The food method can be applied at a grocery store, or at a food shelf or the like. The method can also be adapted for use in online shopping at an online grocery store. The idea is to help and incentivize the consumer to make better food choices. The incentives, such as an amount of credit or a discount, will be higher when the food in the person's cart is healthier.

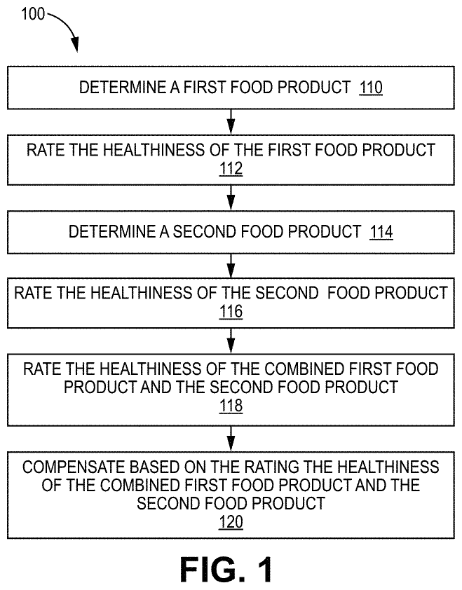

FIG. 1 is a flow diagram of a method 100 for incentivizing the purchasing of healthy food choices, according to one example embodiment. The basic method 100 includes determining a first food product 110 and rating the healthiness of the first food product 112. The method also includes determining a second food product 114 and rating the healthiness of the first food product 116. The healthiness of the combined first food product and the second food product 118 is then determined. Compensation is then determined based on the rating the healthiness of the combined first food product and the second food product 120. As mentioned, this method could be employed without a computer.

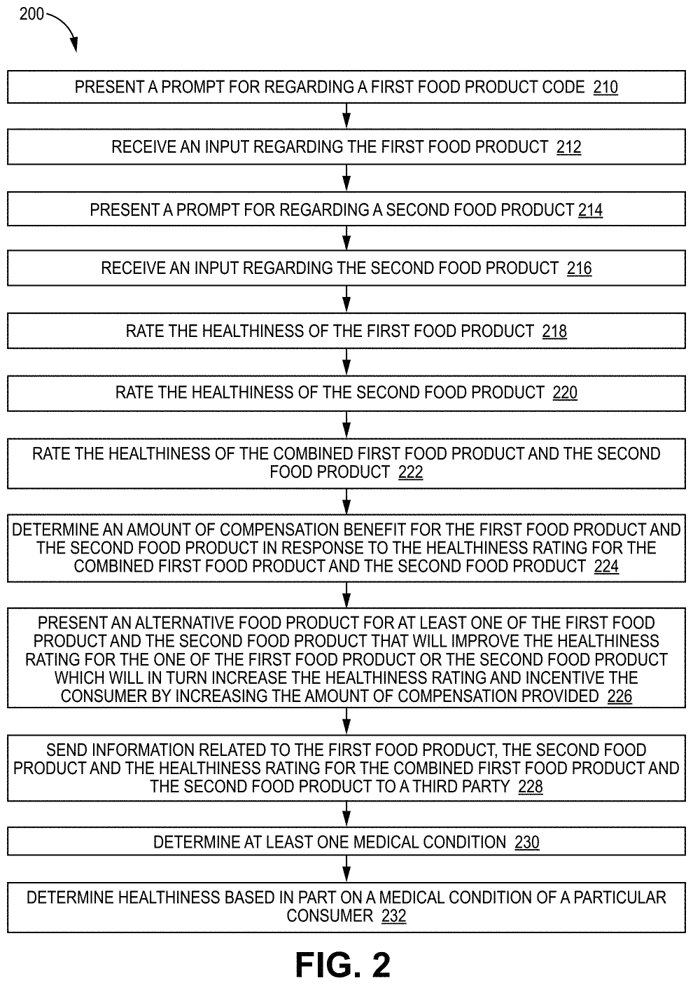

In another embodiment, a computer 2000 is used to enable a method. Any type of computer could be used. For example, the computer used could be a desktop, laptop, notebook, or even a smartphone, or the like. FIG. 2 is a flow diagram of another method 200 for incentivizing the purchasing or consuming of healthy food choices, according to another example embodiment, such as a computerized method 200. The computerized method 200 for monitoring food purchasing includes presenting a prompt for regarding a first food product code 210, receiving an input regarding the first food product 212 and rating the healthiness of the first food product 218. The computerized method 200 does the same for a second food product. More specifically, the computerized method 200 includes presenting a prompt for regarding a second food product 214, receiving an input regarding the second food product 216 and rating the healthiness of the second food product 220. The computerized method also includes rating the healthiness of the combined first food product and the second food product 222.

The computerized method 200 further includes determining an amount of compensation benefit for the first food product and the second food product in response to the healthiness rating for the combined first food product and the second food product 224. The compensation benefit will be higher for a more positive healthiness rating for the combined first food product and the second food product. In one embodiment, the computerized method 200 further includes presenting an alternative food product for at least one of the first food product and the second food product 226. The alternative food product is presented as an alternate choice that will improve the healthiness rating for the one of the first food product or the second food product which will in turn increase the healthiness rating and incentivize the consumer by increasing the amount of compensation they receive since the healthiness rating of the alternative food product and the remaining food product will be higher compared to the healthiness rating for the combined first food product and the second food product.

In some embodiments, the method 200 also includes sending information related to the first food product, the second food product and the healthiness rating for the combined first food product and the second food product to a third party 228. The third party could be a food provider or a health care or health plan professional or a state official working to incentivize people to make more healthy choices. In yet another embodiment, the healthiness rating can be based in part on a medical condition of a particular consumer 232. The healthiness rating for the combination of the first product and the second product having a first value when computed for a first condition and having a second value when computed for a second condition. The method 200 would then further comprise determining at least one medical condition 230. In one example embodiment, a prompt is presented for a first condition, and an input is received for the first condition. A prompt is also presented for a second condition, and an input is received for the second condition. This would essentially be a gathering of information on the condition or conditions, such as one or more medical conditions, that a food consumer might have. Knowing the conditions, the healthiness rating can rate the foods selected based on the one or more conditions. It should be noted that healthiness can change based on the medical condition or conditions associated with an individual. Further, it should be noted that the method 200 can accommodate a plurality of conditions. Some folks may have more than one condition which needs to be taken into consideration when ranking the food choices.

The above methods 100, 200 can be implemented as a software platform that makes it easy for people to think about, access, purchase and pay for food. The software platform can connect people to: food purchasing retailers, grocery/food delivery and/or pick up companies and/or services, and/or donor partners to secure food purchasing subsidies. The software instruction set combined with computer hardware can form modules that perform the methods discussed above. The modules, it should be pointed out, can be computer hardware, computer software or a combination of the above.

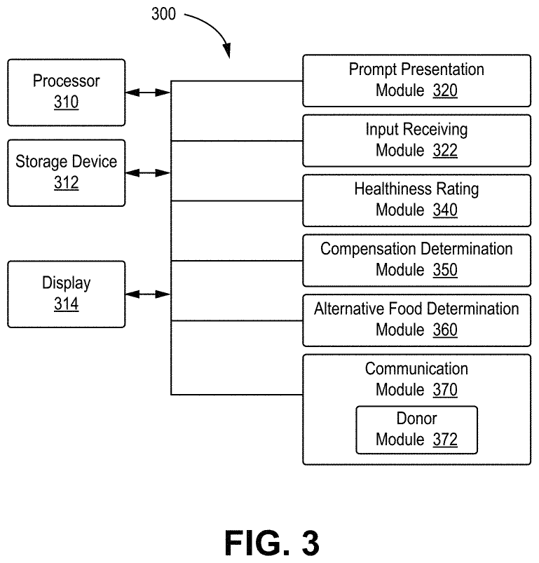

FIG. 3 is a schematic view of a computer system 300 for determining compensation based on food choices, according to an example embodiment. The computer system 300 for determining compensation based on food choices includes a processor 310, and a storage device 312, and a display 314 communicatively coupled to the processor 310. The computer system 300 includes a number of modules that can be computer software, or computer hardware or a combination of hardware and software. The computer system 300 includes a prompt presentation module 320, an input receiving module 322, and a healthiness rating module 340. Each of these modules 320, 322, 340 is communicatively coupled to the processor 310 and the storage device 312. The prompt presentation module 310 presents a first prompt for reading a first food product code, and a second prompt for reading a second food product code. The input receiving module 322 receives a first input regarding the first food product, and a second input regarding the second food product. The healthiness rating module 340 rates the healthiness of the first food product, the second food product; and a combination of the first food product and the second food product. In operation, the computer system 300 can accommodate a plurality of food products, such as an entire shopping cart of food items. The computer system 300 for compensation based on food choices further comprises a compensation determination module 350 for determining an amount of compensation benefit for the first food product and the second food product in response to the healthiness rating for the combined first food product and the second food product. The compensation might be an amount of dollars off on a shopping cart of items, credit given on a governmental credit card used to purchase food or the like. The compensation benefit will be higher for a more positive healthiness rating for the combined first food product and the second food product.

The computer system, in further embodiments, can include an alternative food determination module 360. The alternative food determination module 360 presents an alternative food product for at least one of the first food product and the second food product. The alternative food or substitute food will improve the healthiness rating for the one of the first food product or the second food product and the alternative food product when compared to the healthiness rating for the combined first food product and the second food product. The computer system 300 also has a communications module 370 for sending information related to the first food product, the second food product and the healthiness rating for the combined first food product and the second food product to a third party. In still other embodiments, the compensation determination module 360 includes determination of the healthiness rating based in part on a medical condition or medical conditions. For example, the healthiness rating for the combination of the first food product and the second food product having a first value when computed for a first condition and having a second value when computed for a second condition. In such an embodiment, the prompt presentation module 320 presents a prompt for a first condition, and a prompt for a second condition. Furthermore, the input receiving module 322 receives an input for the first condition, and an input for the second condition.

The food healthiness rating module system 340 includes an algorithm which is based on the nutritional value for selected food items. It is contemplated that a food consumer would select a plurality of items and any that the consumer wishes to receive a benefit for would be rated. The food rating system algorithm or algorithms can be based on evidenced-based food guidelines for a healthy lifestyle, such as those produced by the World Health Organization, or governmental bodies such as the USDA (United States Department of Agriculture) or the CDC (Centers for Disease Control) or the like. In another embodiment, the rating system is based on customized evidence-based food guidelines by underlying health or medical condition or a diagnosis, such as diabetes, cardiovascular disease, gastrointestinal disease, or any other disease. In still another embodiment, the food rating system can be customized by a diet preference. For example, the food rating system could be based on a specific diet such as keto, gluten free, Mediterranean, or the like. The output or rating indicator can be a color coding, such as green, yellow, red; or a numerical rating; or a letter rating; or an emoji icon; or any other rating indicator. It is contemplated that a cart full of food items would be rated. The individual ratings for food items would then be combined in some way to rate the entire shopping cart. Alternatives could be suggested. The shopping cart could be rated while in the store or as each item is placed in the cart so that alternatives could be presented that are in the same area of a store. If shopping online, the cart could also be rated as food items are selected so that any alternatives could be presented while the consumer is thinking about the food item.

The communication module 370 includes a donor module 372 which includes a customizable donor software platform that makes it easy for donors to know and track where, when, and how their contributions (money and/or other goods and/or services, etc.) are being used. The customizable donor software platform can, but is not limited to, allow donors to select the following categories so specify where, when and how they want their contributions to be used. For example, a donor Specific geographic location, such as a particular community, inner city, rural communities, zip code/s, school district, or the like. Donors can also specify particular demographics, such as single parents, elderly, children, veterans, and the like. The donors can also designate specific times of contributions such as real-time notification of a need, weekly, monthly, quarterly, annually, and the like.

Options for the compensation determination module 350 include donation-based subsidies to make healthy food more affordable. Donation based subsidies can be, but are not limited to, offering subsidies based on the food rating system module. Of course, this subsidy is a form of compensation determined by an algorithm, such as the ratio of healthy food being purchased, an average of the healthiness ratings, or a weighted average of the healthiness ratings. Other factors may be used to determine compensation or an amount of a subsidy or benefit to the person purchasing the food.

The communication module 370 can be used to share actionable data with third parties, such as with health plans, health systems, schools and/or school districts, community-based organizations, government agencies and/or other partners and the like. The goal is to aid in their efforts to help people achieve their best health.

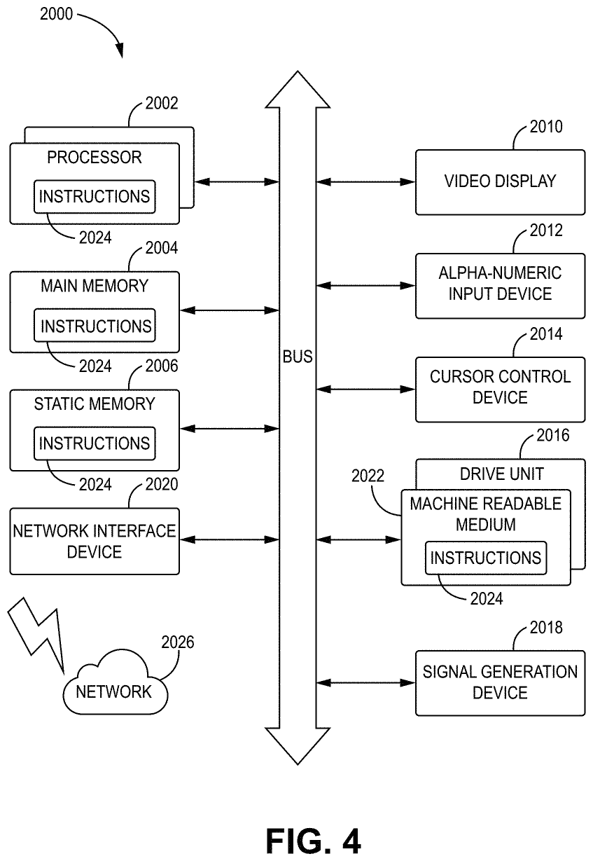

FIG. 4 shows a diagrammatic representation of a computing device for a machine in the example electronic form of a computer system 2000, within which a set of instructions for causing the machine to perform any one or more of the methodologies discussed herein can be executed or is adapted to include the apparatus for generating radiation reports as described herein. In various example embodiments, the machine operates as a standalone device or can be connected (e.g., networked) to other machines. In a networked deployment, the machine can operate in the capacity of a server or a client machine in a server-client network environment, or as a peer machine in a peer-to-peer (or distributed) network environment. The machine can be a personal computer (PC), a tablet PC, a set-top box (STB), a Personal Digital Assistant (PDA), a cellular telephone, a portable music player (e.g., a portable hard drive audio device such as a Moving Picture Experts Group Audio Layer 3 (MP3) player, a web appliance, a network router, a switch, a bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 2000 includes a processor or multiple processors 2002 (e.g., a central processing unit (CPU), a graphics processing unit (GPU), arithmetic logic unit or all), and a main memory 2004 and a static memory 2006, which communicate with each other via a bus 2008. The computer system 2000 can further include a video display unit 2010 (e.g., a liquid crystal display (LCD) or a cathode ray tube (CRT)). The computer system 2000 also includes an alphanumeric input device 2012 (e.g., a keyboard), a cursor control device 2014 (e.g., a mouse), a disk drive unit 2016, a signal generation device 2018 (e.g., a speaker) and a network interface device 2020.

The disk drive unit 2016 includes a computer-readable medium 2022 on which is stored one or more sets of instructions and data structures (e.g., instructions 2024) embodying or utilized by any one or more of the methodologies or functions described herein. The instructions 2024 can also reside, completely or at least partially, within the main memory 2004 and/or within the processors 2002 during execution thereof by the computer system 2000. The main memory 2004 and the processors 2002 also constitute machine-readable media.

The instructions 2024 can further be transmitted or received over a network 2026 via the network interface device 2020 utilizing any one of a number of well-known transfer protocols (e.g., Hyper Text Transfer Protocol (HTTP), CAN, Serial, or Modbus).

While the computer-readable medium 2022 is shown in an example embodiment to be a single medium, the term “computer-readable medium” should be taken to include a single medium or multiple media (e.g., a centralized or distributed database, and/or associated caches and servers) that store the one or more sets of instructions and provide the instructions in a computer readable form. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, tangible forms and signals that can be read or sensed by a computer. Such media can also include, without limitation, hard disks, floppy disks, flash memory cards, digital video disks, random access memory (RAMs), read only memory (ROMs), and the like.

The example embodiments described herein can be implemented in an operating environment comprising computer-executable instructions (e.g., software) installed on a computer, in hardware, or in a combination of software and hardware. Modules as used herein can be hardware or hardware including circuitry to execute instructions. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software programs for implementing the present method(s) can be written in any number of suitable programming languages such as, for example, Hyper text Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Perl, UNIX Shell, Visual Basic or Visual Basic Script, Virtual Reality Markup Language (VRML), ColdFusion™ or other compilers, assemblers, interpreters or other computer languages or platforms.

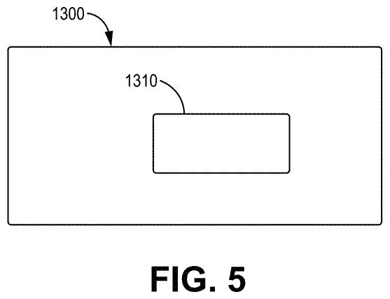

FIG. 5 is a schematic drawing of a machine readable medium 1300 that includes an instruction set 1310, according to an example embodiment. The machine-readable medium 1300 that provides instructions 1310 that, when executed by a machine, cause the machine to perform operations including eliciting and receiving an input to identify a selected investment, and eliciting and receiving an initial offering price for the investment. The machine readable medium 1300 also includes instructions that, when executed by a machine, cause the machine to perform operations that include receiving an input related to prompt displayed on a recycling container, identifying a marketing opportunity associated with the prompt, identifying the source of the received input, and sending the marketing opportunity to the source.

The present disclosure refers to instructions that are received at a memory system. Instructions can include an operational command, e.g., read, write, erase, refresh, etc.; an address at which an operational command should be performed; and the data, if any, associated with a command. The instructions can also include error correction data.

The media 1300 carrying a set of non-transient instructions 1310 for causing a processor associated with a computer system to perform a method that includes presenting a prompt for regarding a first food product code, receiving an input regarding the first food product and rating the healthiness of the first food product. The method carried on the media does the same for a second food product. More specifically, the method includes presenting a prompt for regarding a second food product code, receiving an input regarding the second food product and rating the healthiness of the second food product. The method carried on the media also includes rating the healthiness of the combined first food product and the second food product. The method further includes determining an amount of compensation or benefit for the first food product and the second food product in response to the healthiness rating for the combined first food product and the second food product. The compensation benefit will be higher for a more positive healthiness rating for the combined first food product and the second food product. In one embodiment, the method carried on the media further includes presenting an alternative food product for at least one of the first food product and the second food product. The alternative food product is presented as an alternate choice that will improve the healthiness rating for the one of the first food product or the second food product which will in turn increase the healthiness rating and incentivize the consumer by increasing the amount of compensation they receive since the healthiness rating of the alternative food product and the remaining food product will be higher compared to the healthiness rating for the combined first food product and the second food product. In some embodiments, the method also includes sending information related to the first food product, the second food product and the healthiness rating for the combined first food product and the second food product to a third party. The third party could be a food provider or a health care or health plan professional or a state official working to incentivize people to make more healthy choices. In yet another embodiment, the healthiness rating can be based in part on a medical condition of a particular consumer. The healthiness rating for the combination of the first product and the second product having a first value when computed for a first condition and having a second value when computed for a second condition. The method carried on the media would then further comprise presenting a prompt for a first condition, receiving an input for the first condition, presenting a prompt for a second condition, and receiving an input for the second condition. This would essentially be a gathering of information on the condition or conditions, such as one or more medical conditions, that a food consumer might have. Knowing the conditions, the healthiness rating can rate the foods selected based on the one or more conditions. It should be noted that healthiness can change based on the medical condition or conditions associated with an individual.

In summary, a computerized method for monitoring food purchasing includes presenting a prompt for regarding a first food product code, receiving an input regarding the first food product and rating the healthiness of the first food product. The computerized method does the same for a second food product. More specifically, the computerized method includes presenting a prompt for regarding a second food product code, receiving an input regarding the second food product and rating the healthiness of the second food product. The computerized method also includes rating the healthiness of the combined first food product and the second food product. The computerized method further includes determining an amount of compensation benefit for the first food product and the second food product in response to the healthiness rating for the combined first food product and the second food product, wherein the compensation benefit will be higher for a more positive healthiness rating for the combined first food product and the second food product. In one embodiment, the computerized method further includes presenting an alternative food product for at least one of the first food product and the second food product. The alternative food product is presented as an alternate choice that will improve the healthiness rating for the one of the first food product or the second food product which will in turn increase the healthiness rating and incentivize the consumer by increasing the amount of compensation they receive since the healthiness rating of the alternative food product and the remaining food product will be higher compared to the heathiness rating for the combined first food product and the second food product. In some embodiments, the method also includes sending information related to the first food product, the second food product and the healthiness rating for the combined first food product and the second food product to a third party. The third party could be a food provider or a health care or health plan professional or a state official working to incentivize people to make more healthy choices. In yet another embodiment, the healthiness rating can be based in part on a medical condition of a particular consumer. The healthiness rating for the combination of the first product and the second product having a first value when computed for a first condition and having a second value when computed for a second condition. The method would then further comprise presenting a prompt for a first condition, receiving an input for the first condition, presenting a prompt for a second condition, and receiving an input for the second condition. This would essentially be a gathering of information on the condition or conditions, such as one or more medical conditions, that a food consumer might have. Knowing the conditions, the healthiness rating can rate the foods selected based on the one or more conditions. It should be noted that healthiness can change based on the medical condition or conditions associated with an individual.

A computer system for determining compensation based on food choices a processor, and a storage device communicatively coupled to the processor. The computer system includes a number of modules that can be computer software, or computer hardware or a combination of hardware and software. The computer system includes a prompt presentation module, an input receiving module, and a healthiness rating module. Each of these modules is communicatively coupled to the processor and the storage device. The prompt presentation module presents a first prompt for reading a first food product code, and a second prompt for reading a second food product code. The input receiving module receives a first input regarding the first food product, and a second input regarding the second food product. The healthiness rating module rates the healthiness of the first food product, the second food product and a combination of the first food product and the second food product. The computer system for compensation based on food choices further comprises a compensation determination module for determining an amount of compensation benefit for the first food product and the second food product in response to the healthiness rating for the combined first food product and the second food product. The compensation might be an amount of dollars off on a shopping cart of items, credit given on a governmental credit card used to purchase food or the like. The compensation benefit will be higher for a more positive healthiness rating for the combined first food product and the second food product.

The computer system, in further embodiments, can include an alternative food determination module. The alternative food determination module presents an alternative food product for at least one of the first food product and the second food product. The alternative food or substitute foods will improve the healthiness rating for the one of the first food product or the second food product and the alternative food product when compared to the healthiness rating for the combined first food product and the second food product. The computer system also has a communications module for sending information related to the first food product, the second food product and the healthiness rating for the combined first food product and the second food product to a third party. In still other embodiments, the compensation determination module includes determination of the healthiness rating based in part on a medical condition or medical conditions. For example, the healthiness rating for the combination of the first food product and the second food product having a first value when computed for a first condition and having a second value when computed for a second condition. In such an embodiment, the prompt presentation module presents a prompt for a first condition, and a prompt for a second condition. Furthermore, the input receiving module receives an input for the first condition, and an input for the second condition.

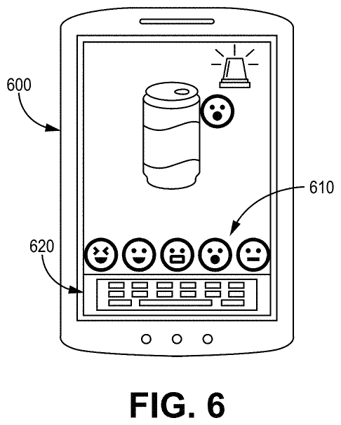

It should be noted that the computer system 300 can be any type of computer such as a laptop computer, notebook computer, desktop computer, point of sale terminal, enterprise system, a smartphone or other smart device. FIG. 6 shows a smartphone 600 that includes a touch screen display 610 that also has a keyboard 620 for inputs. The display 610 shown includes emoji type symbols that could be indicative of healthiness ratings for food. It is anticipated that the above method and computing device could be implemented as an app that runs on a telephone operating system that communicates with virtual server or cloud-based server. The cloud-based server could store data thereon and could also store the software for such a system. In otherwords, the computerized method could be a software as a service application. Many of the computer hardware and software components discussed above could be in the cloud. The software platform could be a web-based system as well. The disclosed method and system is designed to help consumers make better food purchasing decisions and to reward the consumer for making better decisions. Healthier choices are made and the participant merely scans the bar code on the back of any item in any grocery store, even if the participant is not in a grocery store that is part of a network of stores. The input will produce a readout displayed on a device indicative of whether the food choice is a healthy choice for participant. If not, better selections can be displayed for the participant. The program can also be personalized for the participant by entering any medical conditions or allergies into an account for the program. The program will help a participant to make healthier choices that work with the participants unique needs.

In one embodiment, a participant earns Wellness Points for—better grocery shopping. Participants save money by earning earn cash credits in one embodiment. A short time after a trip to the store, participants will get a score for the latest trip to the store. A higher score means the participant purchased many better-for-you items. If there is an item that did not score well, the participant can click on it to find a healthier version. For example, if a participant chose a certain type of cracker, a better-for-you version that is lower in fat, made with whole grains, has a higher fiber content, or is lower in calories and sodium than the crackers you selected is presented as an alternative option.

A recipe section could also be provided with recipes for great-tasting and healthy meals and snacks. Search filters could be provided to find recipe selections to best fit participants and families. The ingredients for a selected recipe can be sent to a shopping list conveniently found on the app, so participants have all the ingredients on the list for the next shopping time. Grocery store “sales circulars” in your area could be collected so that the prices can be compared at various stores before shop to find the best prices available to a participant. Using such a program assures that the latest evidenced-based information for healthy choices is used to assure the better health of the participants. Once a participant registers for the program, instructions are provided over a website for linking to a grocery store loyalty cards or programs. It is contemplated that an app would works at any grocery store regardless of whether the store is part of the health plan program's network. Vendors and suppliers to provide discounts on fruits and vegetables, healthy grains, soups and more.

In one embodiment, a third-party assigns more than 100,000 foods a rating between 0 and 100. Foods rated 100 are considered the most nutritious. Fresh fruits and vegetables always score 100; while sodas and desserts fall in the 0-10 range. (While the average consumer's grocery-basket score is between 30 and 45, a good score is considered above 60. That means you are purchasing more healthy items than non-healthy items.

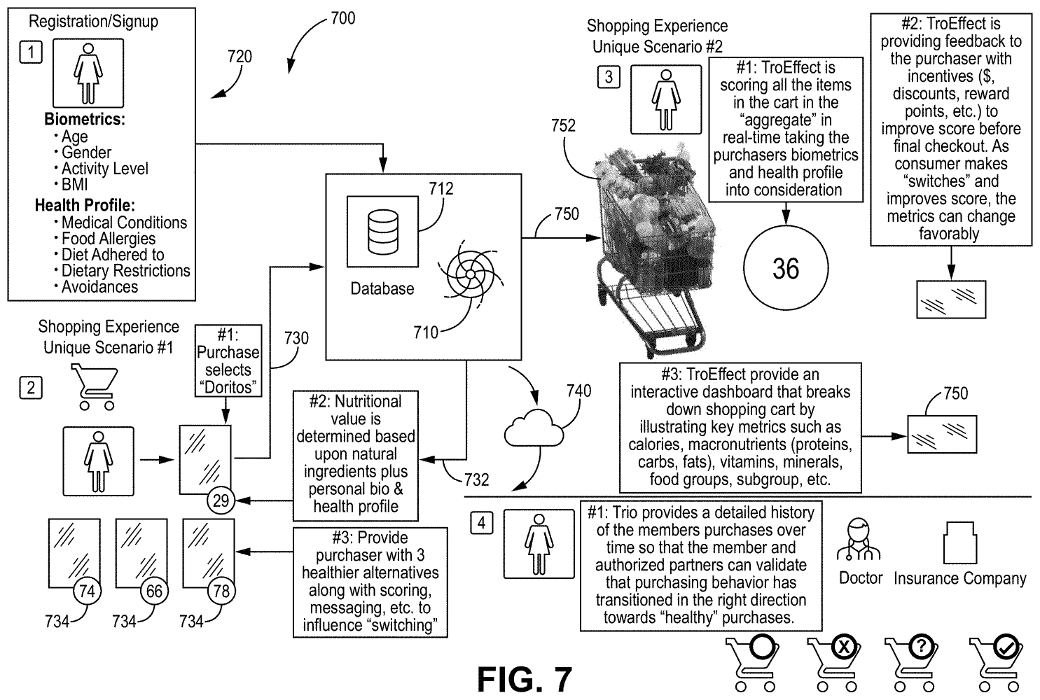

FIG. 7 shows a diagrammatic representation of a system 700 for scoring a collection of food products, according to an example embodiment. The system 700 includes a processor 710 and a storage 712 communicatively coupled to the processor 710. As shown in FIG. 7, the storage 712 is in the form of a database. The system includes a registration signup module 720 where individual inputs biometrics and a health profile are gathered. The information on the biometrics and health profile could be drawn from medical records or provided by an individual or both medical records and an individual could provide the biometrics and health profile. The system 700 also includes an input for food products 730 that are to be added to a collection of foods. The database 712 may include nutritional data on a food as selected or the processor can access such data via a connection to other databases via a connection 740, such as an internet connection or a connection to the cloud or the like. The biometric data is used to set a calorie budget for an individual. The health profile is used to determine the appropriate amounts of various nutrients for a given health profile. The food selected or input at 730 is scored or rated for healthiness based on the biometrics and the health profile. A nutritional value rating is output at output 732. In one embodiment, the nutritional value of other foods is also output at output 732 and alternative foods 734 with higher nutritional value ratings can be presented to the user of the system 700. The alternative foods can be suggested as alternatives to the food selected.

The processor 710 also rates the nutritional value of the foods selected and in the collection or cart of foods 752 and outputs the aggregate nutritional value number for the combination at output 750. Although a traditional cart is shown for the collection of foods 752, it should be noted that one does not need to be shopping for foods to use this system 700. In one example, a person could be setting up a plan for shopping or determining foods that would be better for their health. In another example, a person may be collecting foods at a food shelf or the like. In other words, it is not a requirement nor is it necessary for a person to be actually purchasing foods to use the system. In another example, the person could be shopping online for delivery or later pickup so the cart would be virtual. The nutritional value for the aggregate of all the foods in the collection 752 is also based on the biometrics and the health profile of at least one individual.

The system 700 also includes a display 760. The display can be a dashboard that breaks down the collection of foods by key metrics such as calories, macronutrients, vitamins, minerals, food groups, food subgroups and the like. An overall score for the aggregate in the fool collection 752 can also be displayed. Individual foods in the cart can also be displayed at the display 760. The individual foods can also be broken down and the impact they have on the aggregate score for the collection of foods 752 can also be displayed at the display 760. Alternate foods or substitution suggestions can also be displayed on the display 760. The system 700 also includes an incentive module 770 which will provide incentives for the individual based on the nutritional value of the collection of food products 752.

The system also provides a channel for sharing the information via the cloud or internet 740. Typically, the information about the collection of foods is shared with health professionals for compliance purposes or other third parties that would have an interest in the data. One such third party would be the party providing the incentives for collecting a group of healthy foods.

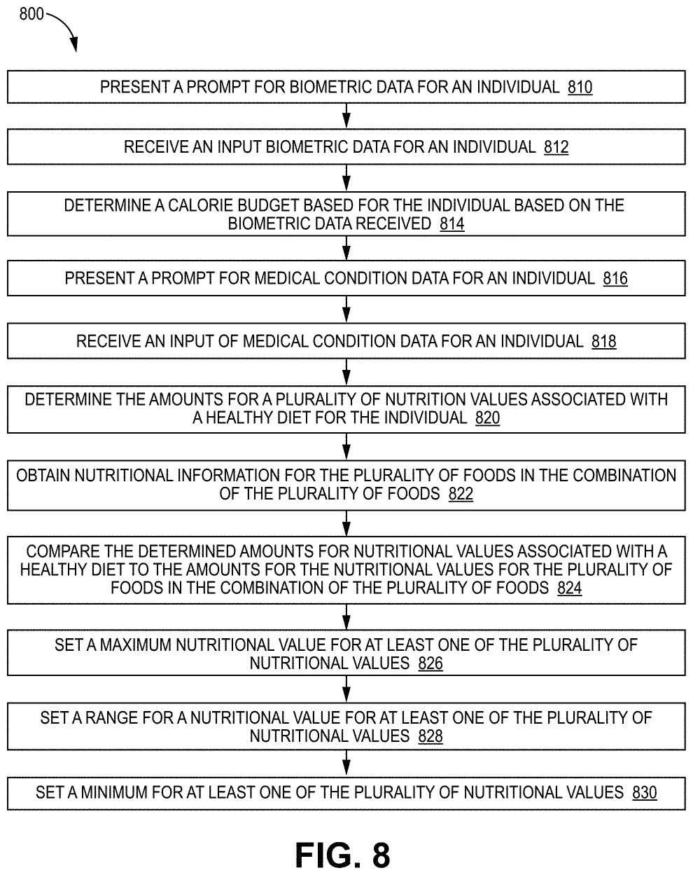

FIG. 8 is a flow diagram of a method 800 for scoring a combination of foods, according to another embodiment of the invention. The method 800 for scoring a combination of a plurality of foods includes presenting a prompt for biometric data for an individual 810 and receiving an input of biometric data for an individual 812. The biometric data includes information about an individual such as their age, gender, activity level, weight, height, body mass index (BMI) and the like. This is stored for further use. For example, the biometric data is used to determine a calorie budget based on the biometric data received for the individual 814. For example, an average calorie budget for a normal healthy individual might be 2000 calories per day. A young, very large football player that works out twice a day will generally need a higher calorie budget so that he has enough energy to get through practices. On the other hand, a senior citizen which only gets moderate amounts of exercise or is totally inactive will need less calories per day.

The method 800 further includes presenting a prompt for a health profile for an individual 816 and receiving an input of health data for an individual 818. The health profile for the individual includes information related to medical conditions, food allergies, dietary restrictions, food or substance avoidances, diets adhered to and the like for the individual. Of course, this is merely an example listing of information for a health profile. It is contemplated that such a list could be much longer and more extensive. The health profile is used to determine the amounts for a plurality of nutritional values associated with a healthy diet for the individual 820. The foods in the combination or collection have to match or substantially match the calorie budget for the individual. Nutritional information for the plurality of foods in the actual combination or actual collection of foods is determined 822. In some instances, the collection of food or combination of foods can be thought of as in a cart. The cart could be an actual cart such as in a market or food shelter. A cart could also be a virtual cart, such as when ordering food over the internet. The determined target amounts for nutritional values associated with a healthy diet are then compared to the actual amounts for the nutritional values for the plurality of foods in the actual combination of the plurality of foods 824. If the actual nutrient values of the plurality of foods in the cart matched the target nutrient values, a perfect score could be attained. For example, a score of 100% could be attained when the actual nutrient values of the collection of actual foods matched the target or ideal values.

Scoring a collection or cart of food items may also include setting a maximum nutritional value for at least one of the plurality of nutritional values 826. If additional amounts of food are selected in that category of foods, then the score of the combination of foods would not increase. For example, kale is generally considered a good food. However, once the maximum for vegetables is met, the cart score will not keep going up but will not increase the scoring of the cart. In some instances, the scoring can go down if the maximum is exceeded. In other instances, a range is set for a nutritional value for at least one of the plurality of nutritional values 828. A top score for the particular nutritional value will require that the nutritional value for the at least one of the plurality of nutritional values be within the range. Scoring a collection or cart of food items may also include setting a minimum for at least one of the plurality of nutritional values 830. In this instance the nutritional content of the collection of food items must include at least the minimum to score well.

As individual food items are added to a collection, the nutrient levels in one or more categories will change. In one embodiment, scoring for the cart is recalculated as each new food product is added to the collection or cart. As foods are added to the cart, the food product impacts the score. This can be referred to as food impact. A food can increase the score of the collection therefore having a positive impact on the scoring. A food can decrease the score of the collection therefore having a negative impact on the scoring. A food can also have no impact.

In some embodiments, alternatives are presented that will have a positive impact on the scoring of the collection. For example, oatmeal might be suggested as an alternative to a sugary cereal food product, such as Frosted Flakes which, in many cases will have a negative food impact on the score of the collection. In still other embodiments, food products not in the cart or collection are scored based on the biometrics and health profile of a person to determine if these products should be suggested as alternatives to food products in the collection or cart.

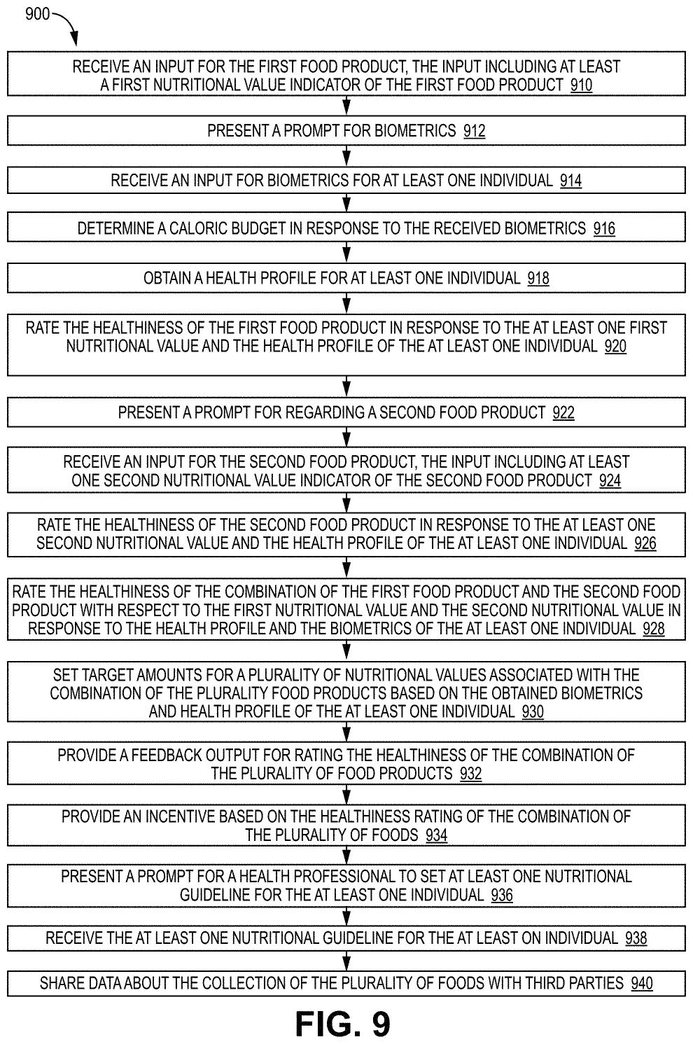

FIG. 9 is a flow diagram for another method 900 of monitoring a collection of a plurality of foods, according to yet another embodiment. The method 900 for monitoring a collection of a plurality of foods includes at least a first food product and a second food product. The method 900 includes receiving an input for the first food product, the input including at least a first nutritional value indicator of the first food product 910. The method also includes presenting a prompt for biometrics 912 and receiving an input for biometrics for at least one individual 914. A calorie budget for the individual is determined in response to the received biometrics 916. A health profile is obtained for the at least one individual 918. The method 900 includes rating the healthiness of the first food product in response to the at least one first nutritional value and the health profile of the at least one individual 920. A prompt is presented for a second food product to be added to the collection 922 and an input is received for the second food product 924. The input for the second food product includes at least one second nutritional value indicator of the second food product. The healthiness of the second food product is rated in response to the at least one second nutritional value and the health profile of the at least one individual 926. The healthiness of the combination of the first food product and the second food product with respect to the first nutritional value and the second nutritional value is also rated in response to the health profile and the biometrics of the at least one individual 928.

The method 900 also may include setting target amounts for a plurality of nutritional values associated with the combination of the plurality food products based on the obtained biometrics and health profile of the at least one individual 930. The target amounts can be set by a health professional, such as a physician, physician's assistant, a nurse or the like. The target amounts can also be set by a dietician or nutritionist. In some instances, the target amounts may be set as default values for various health profile conditions. For example, there may be a dietary restriction for a low salt diet and a default set of nutritional values could be in place for such a dietary restriction. There could also be default values for certain medical conditions, such as diabetes or hypertension or the like. These could be used until a professional reviewed the target amounts to see if the default values needed to be modified for a particular individual. The method 900 may also include presenting a prompt for a health professional to set at least one nutritional guideline for the at least one individual 936 and receiving the at least one nutritional guideline for the at least one individual 938.

The method 900 further comprising providing a feedback output for rating the healthiness of the combination of the plurality of food products 932. When the method is computerized, the feedback output for rating the healthiness of the combination of the plurality of food products is in substantially in real time. In other words, as food products are selected and put in the collection or the cart, the nutritional values of the newly added food product is added to the nutritional values of all or most of the food products in the cart before the recent addition. Scoring or rating is run for the new combination and feedback is provided as a new rating or new score. The rating or scoring may not change instantly but will change fairly quickly so that the effect on the rating can be seen. In other words, the rating of the healthiness of the combination of the plurality of food products is updated as another food product is added to the collection of a plurality of foods. If a food product in the collection or the cart is negatively impacting the healthiness rating of the combination of the plurality of food products, an alternative food product for the first food product can be suggested which generally would increase the healthiness rating of the combination of the plurality of food products. The suggestion is not limited to the last food product selected for the combination of food products but can be for any of the plurality of food products in the collection. It should be noted that the rating of the collection of the food products is dynamic or changeable. In other words, something that was placed in the cart or collection that was labeled or discussed as a cautionary choice, could become something to avoid as other food products and their nutritional values are added to the plurality of food products in the collection. One feedback then would be to remove these food products or replace them with a suggested alternative to raise the rating or score of the collection or cart. Generally, the food products which are suggested for addition to the combination of the plurality of food products will increase the healthiness rating of the combination. Of course, in some embodiments, feedback output for rating the healthiness of the combined first food product and the second food product is displayed on a screen. Feedback on a screen can include a numerical display of the overall rating of the combination of the plurality of foods. Certain foods can also be displayed with individual healthiness ratings. These could be displayed for each of the foods currently in the combination. Other foods which might be presented as alternatives, could also be displayed for feedback purposes. Another display for food products could be simply a plus or a minus for food products with an overall number for the collection of food products. In still another embodiment, the display may use colors to signify the healthiness of food. For example, red could be used to denote that a particular food is bad in the combination or would be a bad addition to the combination. Green could be used to feedback positive food products in the cart or positive additions. Yellow could be used for cautionary food products. In still another embodiment, the numbers used could be food impact numbers. In other words, if added to the cart the food impact number might raise the rating of the combination by a point or lower the combined healthiness rating by two points, for example.

The method 900 wherein obtaining a health profile for at least one individual includes a medical condition, such as hypertension, diabetes, lactose intolerance or the like. The health profile for at least one individual can include a dietary restriction. The health profile could also include a diet the individual prefers to adhere to. Other health profile information can include food allergies, foods to avoid, and the like.

The method 900 for monitoring a collection of a plurality of foods can also include providing an incentive based on the healthiness rating of the combination of the plurality of foods 934. The incentives could be food coupons for future purposes or a rebate available from a third party such as a governmental body or an insurance company, such as the individuals health insurance provider. The incentives could be any kind of incentive including money, or gifts or other awards. The incentives could also be displayed while the plurality of foods for the collection or cart is being selected. The incentives might also accompany a suggestion to place an alternative food in the collection or cart. Of course, the final collection of the plurality of foods would, in some embodiments, be the basis for a final incentive.

The method 900 could also share data about the collection of the plurality of foods with third parties 940. Of course, individual consent would have to be obtained. The third parties could be those that are providing incentives for a good healthiness score or rating on a combination of a plurality of foods. Other third parties may also be interested in the data gathered for one or more individuals. One example would be health care professionals or dieticians that would have an interest in compliance of the individual.

The media 1300, shown in FIG. 5, carries a set of non-transient instructions 1310 for causing a processor associated with a computer system to perform a method 900 and includes instructions presenting a prompt for a first food product, receiving an input for the first food product, the input including at least a first nutritional value indicator of the first food product. The instructions 1310 also cause the processor to present a prompt for biometrics and receive an input for biometrics for at least one individual. Using the biometrics, a calorie budget is determined which is based on the received biometrics. Also obtained is a health profile for the at least one individual. The instructions then cause the processor to rate the healthiness of the first food product in response to the at least one first nutritional value and the health profile of the at least one individual. A prompt is presented for a second food product and an input is received for the second food product. The input includes at least one second nutritional value indicator of the second food product. The second food product is rated for healthiness in response to the at least one second nutritional value and the health profile of the at least one individual. The combination of the first food product and the second food product is rated for healthiness with respect to the first nutritional value and the second nutritional value in response to the health profile and the biometrics of the at least one individual. The instructions can further cause the processor to send prompts and get input needed to set target amounts for a plurality of nutritional values associated with the combination of the plurality food products based on the obtained biometrics and health profile of the at least one individual. In still further embodiments, the set of non-transient instructions cause a processor associated with a computer system to provide a feedback output for rating the healthiness of the combination of the plurality of food products, which includes the first food product and the second food product. The feedback output can be provided in substantially real time. In other words, as food products are added to the collection of the plurality of foods, the rating of the healthiness is recalculated and output.

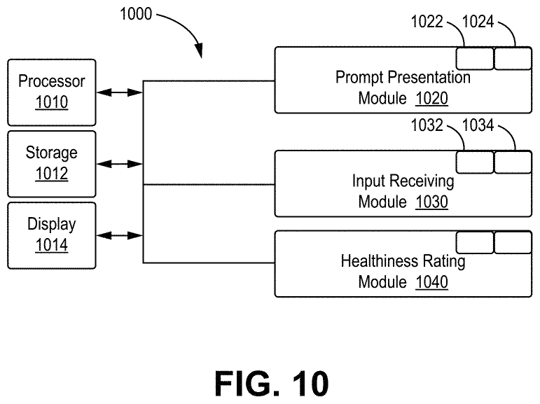

FIG. 10 is another schematic diagram of a computer system 1000, according to an example embodiment. The computer system 1000 includes a processor 1010, a storage device 1012 communicatively coupled to the processor 1010 and a prompt presentation module 1020 also communicatively coupled to the processor 1010 and the storage device 1012. The prompt presentation module presents a first prompt for biometrics of an individual 1022 and a second prompt for a health profile of an individual 1024. The computer system also includes an input receiving module130 communicatively coupled to the processor 1010 and the storage device 1012 the input receiving module 1030 for receiving a first input regarding the biometrics of an individual 1032 and a second input regarding the health profile of an individual 1034. The computer system 1000 also includes aa healthiness rating module 1040 communicatively coupled to the processor 1010 and the storage device 1012, and the input receiving device 1030. The healthiness rating module 1040 determines a calorie budget for an individual in response to the first input and determines a combination of nutritional values in response to the health profile of the individual. The combination of nutritional values is associated with a healthy diet. The healthiness rating module 1040 also includes an actual food nutritional value determination module1050 for determining actual nutritional values of a combination of a plurality of foods actually selected. The computer system 1000 also includes a comparator 1060 for comparing the determined high combination of nutritional values to the actual nutritional values of the plurality of foods actually selected. It outputs a number of the determined high combination of nutritional values compared to the actual nutritional values. In one embodiment, the actual determination is divided by the high determined value for a combination of food nutritional values to yield a percentage. There are other ways to compare values that are also contemplated.

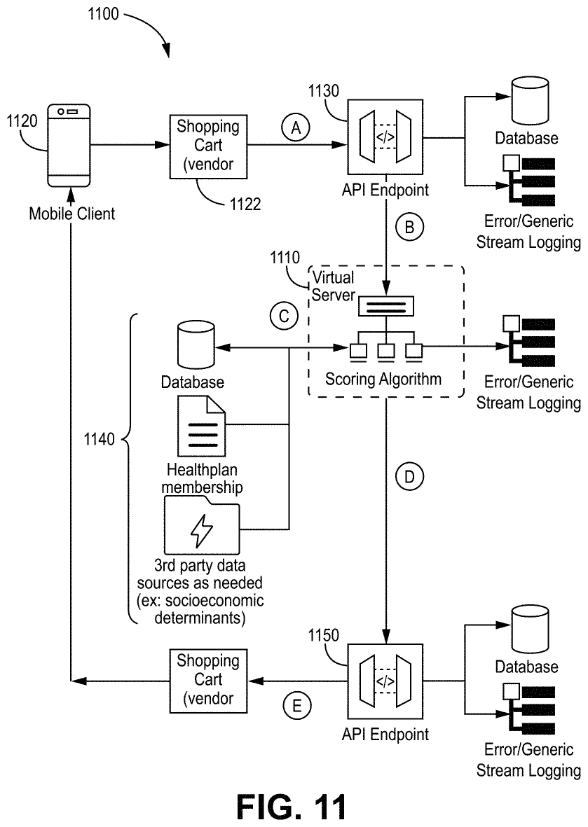

FIG. 11 is a flow diagram for data used by a computer system 1100, according to an example embodiment. The computer system 1100 in this embodiment is a cloud based virtual server 1110. The virtual server 1110 accesses a processor 1112 for scoring or rating the healthiness of a collection of foods. A mobile client 1120 inputs a collection of foods or shopping cart as food products are selected and placed into the cart 1122. This is forwarded to a first API endpoint 1130 as API data. Other data is also input to the first API endpoint 1130 such as login information, secure API tokens, REST API, JSON payload and the like. The incoming data is then input to the start of the processor that determines healthiness ratings or scoring. External data sources 1140 provide food nutrient data, insurance plan eligibility data, user metadata, other modifiers and the like to the processor and virtual server 1110. A final payload which includes the healthiness rating for the collection of foods, subsidies and identifiers for each cart item and the like is output from the virtual server 1110 to a second API endpoint 1150. The second API endpoint 1150 delivers outbound data to the mobile client 1120. The outbound data also may include suggestions and summary data for graphs and the like.

### SUMMARY

The disclosed solution includes:
1. Software platform—a responsive design client/server application using the cloud and portable devices that makes it easy for people to think about, access, purchase and pay for food:
   a. The software platform can, but is not limited to, connect people to: food purchasing retailers, grocery/food delivery and/or pick up companies and/or services, and/or donor partners to secure food purchasing subsidies.
2. Food rating system for food purchasing, education, incentivizing, and monitoring based upon, but not limited to, the following:
   a. Quality, as defined by nutrient content, of the items selected and can be determined by, but not limited to, evidence-based food guidelines for a healthy lifestyle, i.e., World Health Organization, USDA, CDC, etc.
   b. Quality, as defined by nutrient content, of the items in relation to the consumer's biometrics (age, gender, activity, BMI, etc.) AND the consumers Medical Profile (medical conditions, diseases, disorders, diets, restrictions, avoidances, etc.)
   c. Quantity of the items in relation to evidence based nutritional recommendations for a balanced diet
   d. Continuous real-time monitoring of shopping cart & feedback to the consumer provides information, incentives, and motivation for consumer to continuously attempt to improve shopping cart score.
   e. The real-time review and assessment of a specific item immediately after selection provides purchaser with information on the quality of the item selected and suggestions for healthier selections (where applicable). This educates and influences consumers to consider de-selecting items in cart and replacing them with healthier alternatives.
   f. The food rating system or systems can include but is not limited to:
      i. Color coding
      ii. Numerical rating
      iii. Letter ratings
      iv. Emoji icon ratings
      v. And any other rating indicator/s
   g. The aggregation of items, education, evaluation, incentives, continuous real-time scoring and feedback to the consumer, health plan, healthcare provider, and the like.
   h.
3. Actionable data that can be shared with health plans, health systems, schools and/or school districts, community-based organizations, government agencies and/or other partners, to aid in their efforts to help people achieve their best health. Data can include but is not limited to:
   a. Share biometric data collected overtime with health plans/providers. Observing improvements in biometrics like, but not limited to, weight, body mass index (BMI), blood sugar, hemoglobin A1C (HbA1C), etc. demonstrates the value of Trio Effect and provides additional insight into biometric trends in their member/patients.
   b. Sharing food purchasing trend information of users of this invention. This can be compared to medical costs and outcomes to determine the impact of the disclosed solution.
   c. Additional reporting can include, but is not limited to:
      i. Food insecurity improvement adherence
      ii. Nutritional data
      iii. Shopping habits/trends
      iv. Utilization reports
      v. Anonymous member aggregation reports (various)
4. Donation based subsidies to achieving best health affordable:
   a. Donation based subsidies can be, but are not limited to, offering subsidies based on the food rating system/s (algorithm/s) ratio of healthy food being purchased.
5. Customizable donor software platform that makes it easy for donors to know and track where, when, and how their contributions (money and/or other goods and/or services, etc.) are being used.
   b. The customizable donor software platform can, but is not limited to, allow donors to select the following categories so specify where, when and how they want their contributions to be used:
      i. Specific geographic location, i.e., own community, inner city, rural communities, zip code/s, school district, etc.
      ii. Specific demographics, i.e., single parents, elderly, children, veterans,
      iii. Specific times of contributions, i.e., real-time notification of a need, weekly, monthly, quarterly, annually, etc.

This has been a detailed description of some exemplary embodiments of the invention(s) contained within the disclosed subject matter. Such invention(s) may be referred to, individually and/or collectively, herein by the term “invention” merely for convenience and without intending to limit the scope of this application to any single invention or inventive concept if more than one is in fact disclosed. The detailed description refers to the accompanying drawings that form a part hereof and which shows by way of illustration, but not of limitation, some specific embodiments of the invention, including a preferred embodiment.

These embodiments are described in sufficient detail to enable those of ordinary skill in the art to understand and implement the inventive subject matter. Other embodiments may be utilized and changes may be made without departing from the scope of the inventive subject matter. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for monitoring a collection of a plurality of foods including at least a first food product and a second food product comprising:
presenting a prompt for a first food product;
receiving an input for the first food product, the input including at least a first nutritional value indicator of the first food product;
presenting a prompt for biometrics;
receiving an input for biometrics for at least one individual;
determining a calorie budget in response to the received biometrics;
obtaining a health profile for the at least one individual;
rating the healthiness of the first food product in response to the at least one first nutritional value and the health profile of the at least one individual;
presenting a prompt for regarding a second food product;
receiving an input for the second food product, the input including at least one second nutritional value indicator of the second food product;
rating the healthiness of the second food product in response to the at least one second nutritional value and the health profile of the at least one individual; and
rating the healthiness of the combination of the first food product and the second food product with respect to the first nutritional value and the second nutritional value in response to the health profile and the biometrics of the at least one individual.

2. The method for monitoring a collection of a plurality of foods of claim 1 further comprising setting target amounts for a plurality of nutritional values associated with the combination of the plurality food products based on the obtained biometrics and health profile of the at least one individual.

3. The method for monitoring a collection of a plurality of foods of claim 1 further comprising providing a feedback output for rating the healthiness of the combination of the plurality of food products, that includes the first food product and the second food product.

4. The method for monitoring a collection of a plurality of foods of claim 3 wherein the method is computerized, the method further comprising providing the feedback output, in substantially real time, for rating the healthiness of the combination of the plurality of food products.

5. The method for monitoring a collection of a plurality of foods of claim 4 wherein the rating of the healthiness of the combination of the plurality of food products is updated as an other food product is added to the collection of a plurality of foods.

6. The method for monitoring a collection of a plurality of foods of claim 4 wherein providing the feedback output for rating the healthiness of the combination of the plurality of food products further includes suggesting an alternative food product for the first food product which would increase the healthiness rating of the combination of the plurality of food products.

7. The method for monitoring a collection of a plurality of foods of claim 4 wherein providing the feedback output for rating the healthiness of the combination of the plurality of food products further includes suggesting a food product to add to the combination of the plurality of foods which would increase the healthiness rating.

8. The method for monitoring a collection of a plurality of foods of claim 3 wherein providing the feedback output for rating the healthiness of the combined first food product and the second food product is displayed on a screen.

9. The method for monitoring a collection of a plurality of foods of claim 1 wherein obtaining a health profile for at least one individual includes a medical condition.

10. The method for monitoring food purchasing of claim 1 wherein obtaining a health profile for at least one individual includes a dietary restriction.

11. The method for monitoring a collection of a plurality of foods of claim 1 further comprising providing an incentive based on the healthiness rating of the combination of the plurality of foods.

12. The method for monitoring food a collection of a plurality of foods of claim 1 further comprising:
presenting a prompt for a health professional to set at least one nutritional guideline for the at least one individual; and
receiving the at least one nutritional guideline for the at least one individual.

13. A method for scoring a combination of a plurality of foods comprising:
presenting a prompt for biometric data for an individual;
receiving an input of biometric data for an individual;
determining a calorie budget based for the individual based on the biometric data received;
presenting a prompt for medical condition data for an individual;
receiving an input of medical condition data for an individual;
determining the amounts for a plurality of nutritional values associated with a healthy diet for the individual;
obtaining nutritional information for the plurality of foods in the combination of the plurality of foods; and
comparing the determined amounts for nutritional values associated with a healthy diet to the amounts for the nutritional values for the plurality of foods in the combination of the plurality of foods.

14. The method for scoring a combination of a plurality of foods of claim 13 wherein the score for the combination is higher when nutritional values of the combination of the plurality of foods as the determined amounts for the plurality of nutritional values are approached.

15. The method for scoring a combination of a plurality of foods of claim 13 further comprising setting a maximum nutritional value for at least one of the plurality of nutritional values wherein the score for the combination remains substantially the same after going over a determined maximum amount for one of the nutritional values of the plurality of nutritional values.

16. The method for scoring a combination of a plurality of foods of claim 13 further comprising setting a maximum nutritional value for at least one of the plurality of nutritional values wherein the score for the combination declines after going over a determined maximum amount for one of the nutritional values of the plurality of nutritional values.

17. The method for scoring a combination of a plurality of foods of claim 13 further comprising setting a range for a nutritional value for at least one of the plurality of nutritional values wherein the score for the combination declines the nutritional value goes beyond the range.

18. A media carrying a set of non-transient instructions for causing a processor associated with a computer system to perform a method comprising:
presenting a prompt for a first food product;
receiving an input for the first food product, the input including at least a first nutritional value indicator of the first food product;
presenting a prompt for biometrics;
receiving an input for biometrics for at least one individual;
determining a calorie budget in response to the received biometrics;
obtaining a health profile for the at least one individual;
rating the healthiness of the first food product in response to the at least one first nutritional value and the health profile of the at least one individual;
presenting a prompt for regarding a second food product;
receiving an input for the second food product, the input including at least one second nutritional value indicator of the second food product;
rating the healthiness of the second food product in response to the at least one second nutritional value and the health profile of the at least one individual; and
rating the healthiness of the combination of the first food product and the second food product with respect to the first nutritional value and the second nutritional value in response to the health profile and the biometrics of the at least one individual.

19. The media carrying a set of non-transient instructions for causing a processor associated with a computer system to perform a method of claim 18 further comprising setting target amounts for a plurality of nutritional values associated with the combination of the plurality food products based on the obtained biometrics and health profile of the at least one individual.

20. The media carrying a set of non-transient instructions for causing a processor associated with a computer system to perform a method of claim 18 further comprising providing a feedback output for rating the healthiness of the combination of the plurality of food products, that includes the first food product and the second food product.

21. The media carrying a set of non-transient instructions for causing a processor associated with a computer system to perform a method of claim 20 further comprising providing the feedback output, in substantially real time, for rating the healthiness of the combination of the plurality of food products.

22. The media carrying a set of non-transient instructions for causing a processor associated with a computer system to perform a method of claim 20 wherein the rating of the healthiness of the combination of the plurality of food products is updated as an other food product is added to the collection of a plurality of foods.

23. A computer system for compensation based on food choices comprising:
a processor;
a storage device communicatively coupled to the processor;
a prompt presentation module communicatively coupled to the processor and the storage device for presenting
a first prompt for biometrics of an individual; and
a second prompt for a health profile of an individual;
an input receiving module communicatively coupled to the processor and the storage device, the input receiving module for receiving
a first input regarding the biometrics of an individual; and
a second input regarding the health profile of an individual;
a healthiness rating module communicatively coupled to the processor and the storage device, and the input receiving device, the healthiness rating module
determining a calorie budget for an individual in response to the first input, and
determining a combination of nutritional values in response to the health profile of the individual, the combination of nutritional values associated with a healthy diet; and
a food nutritional value determination module for determining actual nutritional values of a combination of a plurality of foods actually selected; and
a comparator for comparing the determined combination of nutritional values to the actual nutritional values of the plurality of foods actually selected.
